(19) United States
(12) Patent Application Publication
(10) Pub. No.: US 2011/0110819 A1
(43) Pub. Date: May 12, 2011

(54) Sanitization process for objects and sanitization chamber

(76) Inventors: Mark Stuart Allen, London (CA); David Gary Fowler, St. Thomas (CA); Gordon Craig Combe, London (CA); Stephen Fabian McNeil, London (CA); Christopher John Arscott, London (CA); Michael George Brock, London (CA)

(21) Appl. No.: 12/591,319

(22) Filed: Nov. 16, 2009

(30) Foreign Application Priority Data
Nov. 6, 2009 (CA) 2684767

(51) Int. Cl. A61L 2/20

(52) U.S. Cl. 422/29; 422/37; 422/129

(57) ABSTRACT

The present invention provides a process for sanitizing an object. The process comprises: (i) placing the object to be sanitized in a substantially enclosed environment, the environment being at atmospheric pressure when the object is placed therein, (ii) providing a gaseous chlorine dioxide vapour in the environment, and (iii) exposing the gaseous chlorine dioxide vapour to the object to sanitize the object. The process includes maintaining a pressure in the environment at substantially atmospheric pressure.

The present invention relates to a sanitization process and a sanitization chamber for carrying out this process, and in particular, to a sanitization process for sanitizing objects, such as electronic devices, and a sanitization chamber for carrying out this process.

### BACKGROUND OF THE INVENTION

Infectious diseases are a major cause of health problems and mortality throughout the world. According to the World Health Organization, infectious diseases caused 14.7 million deaths worldwide in 2002, which is equal to about 25.9% of all deaths in 2002. Lower respiratory infections, such as pneumonias, influenzas and acute bronchitis, were responsible for 3.9 million deaths worldwide in 2002, which is equal to about 6.9% of all deaths in 2002. In the United States and Canada, there is an estimated cost of health care to treat these infectious diseases possibly exceeding $100 billion annually.

Infectious diseases are caused by pathogenic microorganisms or microbes, including viruses, bacteria, fungi, protozoa, parasites and prions, and can be spread, directly or indirectly, from one person to another. It is known that infectious diseases are often spread by contact with contaminated objects. Pathogenic microorganisms are known to contaminate objects commonly found in the home and work environments, such as pens, pencils, staplers, phones, cellular phones, computer keyboards, and TV hand held controllers.

Electronic devices, such as computer keyboards, cellular phones, laptop computers, handheld video game devices, portable audio players, portable video players, and global positioning system devices, have been found to often harbour disease causing microbes including staphylococcus, corynebacterium diphtherine, micrococcus, and bacillus. This is especially common for electronic devices which are designed to be hand-operated. People tend to pass pathogenic and/or disease causing microbes to electronic devices when they come into contact with the electronic device's hand-operated input keys or touch screen.

Staphylococcus, for example, is known to cause a wide variety of infections in humans, from skin infections to pneumonia. As another example, corynebacterium diphtherine is responsible for the upper respiratory tract disease called diphtheria. One type of bacillus species, B. anthracis, is responsible for anthrax. There are even studies which indicate that computer keyboards tend to be contaminated with more harmful bacteria than toilet seats. Studies indicate that some bacteria, including staphylococcus aureus, can survive on computer keyboards for more than twenty-four hours, thus increasing the chances of transmission of the bacteria to a person subsequently using the keyboard. It is often recommended that computer keyboards in the work environment be disinfected on a regular basis, as often as once per day.

The contamination of computer keyboards is especially problematic in hospital environments. With the emerging trend of keeping electronic health records for hospital patients, computers with computer keyboards are often found in patient rooms. The contamination of computer keyboards leads to disease-causing microbes, including antibiotic-resistant microbes, being passed from nurses or doctors to patients who are already suffering from health problems.

The sanitization of electronic devices, such as computer keyboards and cellular phones, is especially difficult because of the delicate electronic circuitry in these objects. Previous methods of sanitization of objects include steam, high heat exposure, ethylene oxide, peroxide and ozone. However, all of these previous methods can potentially cause damage to the circuitry of electronic devices.

Disinfectant liquid sprays are known for use in sanitizing objects. However, such liquids can cause damage to the circuitry of electronic devices.

Disinfectant wipes are also known to be used for sanitizing objects. However, such wipes cannot reach every surface of the electronic devices, such as the inner surfaces. The electronic device needs to be disassembled, and the individual components wiped. However, such processes for sanitization are slow, cumbersome and can cause damage to the electronic circuitry.

It is known to use chlorine dioxide for a variety of purposes. The most common use for chlorine dioxide is for bleaching wood pulp. Another known use for chlorine dioxide is for the disinfection of drinking water. In the treatment of water, chlorine dioxide is often used as a pre-oxidant prior to the chlorination of drinking water to destroy natural water impurities, and in some cases, is used to replace chlorination completely.

### SUMMARY OF THE INVENTION

It is an object of the present invention to provide a sanitization process for killing microorganisms particularly pathogenic microorganisms, including viruses, bacteria, fungi, protozoa, parasites and prions, which contaminate objects, especially electronic devices, and a sanitization chamber for carrying out this process.

It is another object of the present invention to provide a sanitization process and a sanitization chamber where disease causing microbes do not build up resistance to the sanitization process.

It is a further object of the present invention to provide a sanitization process which does not cause any damage to the object being treated, and in particular, does not damage the electronic circuitry of electronic devices.

Another object of the present invention is to provide a sanitization process and a sanitization chamber which is safe and easy to use.

A further object of the present invention is to provide a sanitization process and a sanitization chamber where the components used are safe and easy to transport.

Yet another object of the present invention is to provide a sanitization process where the components used in this process have a long shelf life.

It is a further object of the present invention to provide a sanitization process which does not result in side chemical reactions that produce harmful disinfection by-products.

In one aspect, the present invention provides a process for sanitizing an object, the process comprising: (i) placing the object to be sanitized in a substantially enclosed environment, the environment being at atmospheric pressure when the object is placed therein, (ii) providing a gaseous chlorine dioxide vapour in the environment, and (iii) exposing the gaseous chlorine dioxide vapour to the object to sanitize the object; including maintaining a pressure in the environment at substantially atmospheric pressure.

In another aspect, the present invention provides a sanitization chamber for sanitizing an object, the sanitization chamber being substantially enclosed, the sanitization chamber comprising: a reaction section where a gaseous chlorine dioxide vapour is generated, and a sanitization section where the object to be sanitized is placed, the object being exposed to the gaseous chlorine dioxide vapour in the sanitization section, a pressure in the sanitization chamber being at atmospheric pressure when the object is placed therein, and the pressure in the sanitization chamber being maintained at substantially atmospheric pressure during the sanitizing of the object.

The sanitization chamber is preferably substantially enclosed such that gases inside the sanitization chamber can pass outside the sanitization chamber to maintain the pressure in the sanitization chamber at substantially atmospheric pressure. The sanitization chamber is not airtight or hermetically sealed, thus allowing gas to pass out. The pressure in the sanitization chamber is preferably maintained so as to not exceed 0.2 psig above atmospheric pressure, more preferably so as to not exceed 0.1 psig above atmospheric pressure.

Preferably, the object being sanitized is an electronic device. The electronic device comprises an electronic circuit. The electronic circuit is made up electronic components connected together by conductors to form a closed path through which an electric current can flow.

The electronic device preferably comprises a hand-operated input for entering or changing information. Examples of a hand-operated input include the keys on a computer keyboard and a touch screen on a portable audio player.

Examples of electronic devices include, but are not limited to, a computer keyboard, a laptop computer, a cellular phone, a handheld video game device, a portable audio player, a portable video player, and a global positioning system device.

The process of the present invention is carried out without causing damage to the electronic circuit. In a preferred embodiment, the electronic circuit continues to operate during the process of the present invention.

Preferably, the gaseous chlorine dioxide vapour is generated by reacting a chlorite source with an acid releasing agent.

The chlorite source is preferably sodium chlorite, potassium chlorite, calcium chlorite, ammonium chlorite, trialkylammonium chlorite or quarternary ammonium chlorite. The acid releasing agent is preferably a carboxylic acid, an anhydride, an acyl halide, a phosphoric acid, a phosphate ester, a trialkylsilyl phosphate ester, a dialkyl phosphate, a poly phosphate, a condensed phosphate, a sulfonic acid, a sulfonic acid ester, a sulfonic acid chloride, a phosphosilicate, a phosphosilicic anhydride, a carboxylate of poly α-hydroxy alcohol, a phosphosiloxane, hydrochloric acid, boric acid, citric acid, malic acid, tartaric acid, or a mineral acid.

In a preferred embodiment, the chlorite source is sodium chlorite and the acid releasing agent is sodium bisulfate. Both the sodium chlorite and the sodium bisulfate are preferably in the form of dry powders.

The dry powder of sodium chlorite and the dry powder of sodium bisulfate are preferably mixed with water, more preferably 100 mL of hot water, to generate the gaseous chlorine dioxide vapour. This reaction to generate the gaseous chlorine dioxide vapour takes place in the reaction section of the sanitization chamber.

The sanitization chamber preferably has a pathway extending between the reaction section and the sanitization section. The gaseous chlorine dioxide vapour is generated as described above in the reaction section. The gaseous chlorine dioxide vapour then travels through the pathway and into the sanitization section where the object is held.

Preferably, the sanitization chamber comprises a base and a cover. The reaction section, the sanitization section and the pathway are formed in the base. When the generating of the gaseous chlorine dioxide vapour begins in the reaction section, the cover is placed over the base to substantially enclose the sanitization chamber.

### BRIEF DESCRIPTION OF THE DRAWINGS

Further aspects and advantages will become apparent from the following description taken together with the accompanying drawings in which:

FIG. 1 is a perspective view of a base of a sanitization chamber in accordance with a preferred embodiment of the present invention.

FIG. 2 is a perspective view of a closed sanitization chamber in accordance with a preferred embodiment of the present invention.

FIG. 3 is view of an open sanitization chamber in accordance with a preferred embodiment of the present invention when being used for sanitizing a computer keyboard and a computer mouse.

FIG. 4 is a view of the sanitization chamber of FIG. 3 when used for sanitizing two telephones.

Throughout all the drawings and the disclosure, similar parts are indicated by the same reference numerals.

### DESCRIPTION OF THE PREFERRED EMBODIMENT

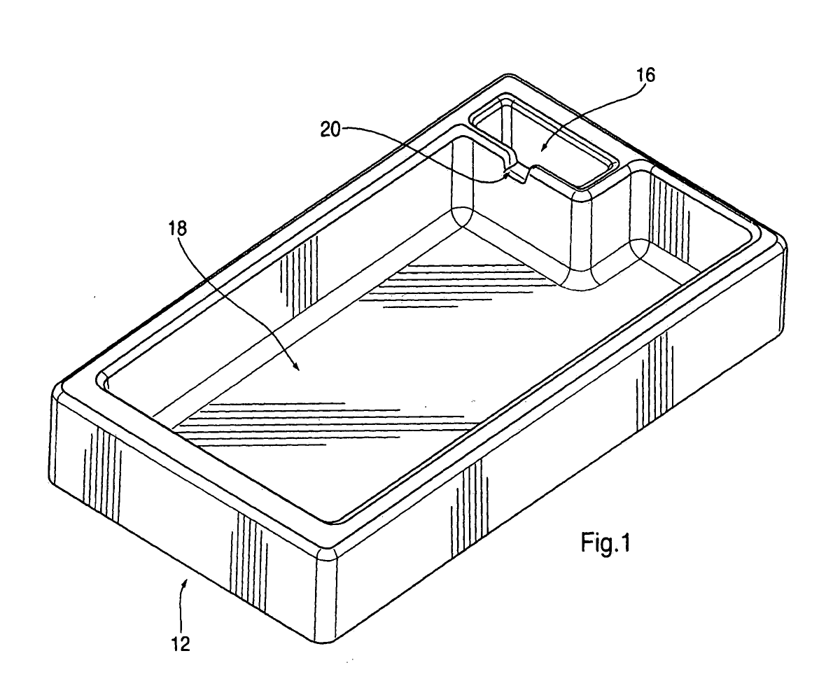

Reference is made to FIGS. 1 to 4 which show a preferred embodiment of the sanitization chamber 10 of the present invention.

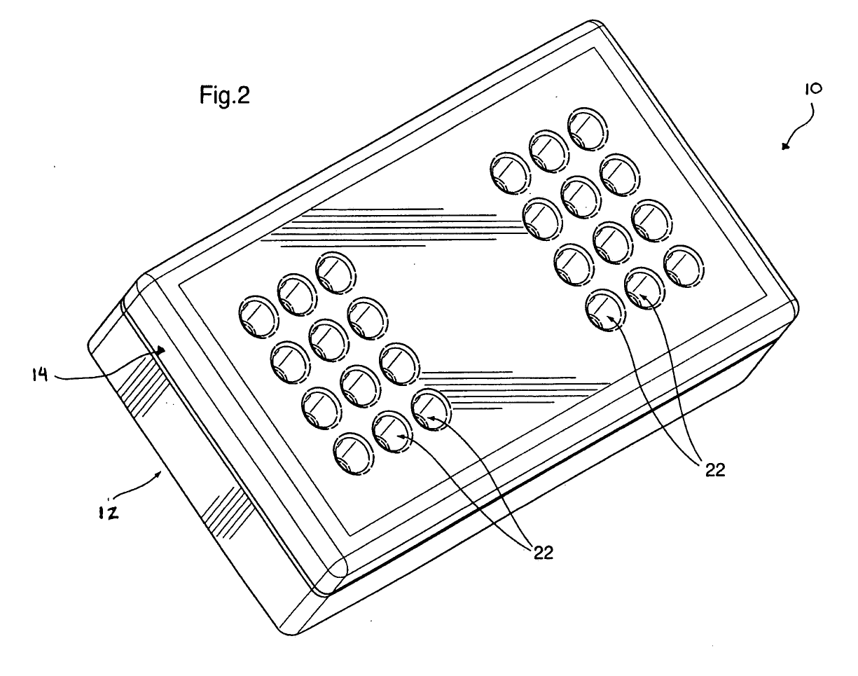

FIG. 1 illustrates the base 12 of the sanitization chamber 10. FIG. 2 illustrates the cover 14 of the sanitization chamber 10 when put over the base 12 to close the sanitization chamber 10. The sanitization chamber 10, including both the base 12 and the cover 14, can be made of any solid material. Preferably, both the base 12 and the cover 14 are made of plastic or steel.

When the base 12 and the cover 14 are made of plastic, they can be manufactured by known methods of vacuum forming. Sheets of plastic are: (i) heated to a forming temperature; (ii) stretched onto a mold having the desired shape of the base 12 or cover 14; and (iii) held against the mold by applying vacuum between the surface of the mold and the plastic sheet.

When the base 12 and the cover 14 are made of steel, these components are preferably of made of stainless steel. The stainless steel base 12 and cover 14 can be made by known methods for steel shaping, including known methods of steel rolling, stamping and deep drawing.

As shown in FIG. 1, the base 12 of the sanitization chamber 10 has a well 16 which is used as a reaction section for forming gaseous chlorine dioxide gas. In the well 16, gaseous chlorine dioxide vapour is produced by reacting a chlorite source with an acid releasing agent. The preferred chlorite source is sodium chlorite and the preferred acid releasing agent is sodium bisulphate. It is also preferred that both the chlorite source and the acid releasing agent be in the form of solid dry powders. In a preferred embodiment, a dry powder of sodium chlorite and a dry powder of sodium bisulphate are mixed with water in the well 16 to form the gaseous chlorine dioxide vapour. The water is preferably hot water.

The well 16 is preferably sized so as to hold a set volume of water. In a preferred embodiment, the set volume of water is about 100 mL. A visible line can be formed or drawn into the side of the well 16 to mark a 100 mL volume.

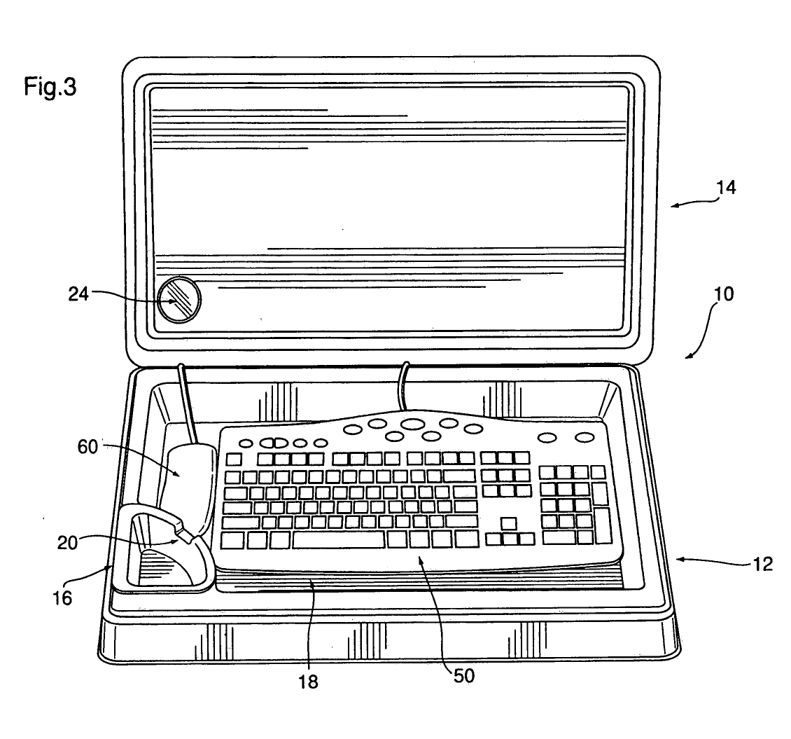

The base 12 also has a sanitization section 18 for holding the object or objects to be sanitized. The sanitization section 18 is preferably sized and shaped so as to fit the particular type of object to be sanitized. For example, as shown in FIG. 3, the sanitization section 18 is of a size that fits a computer keyboard 50 and a computer mouse 60. The computer keyboard 50 and the computer mouse 60 preferably take up at least 70% of the volume of the sanitization section 18, more preferably at least 80% and even more preferably at least 90%. Preferably, the sanitization section 18 is of a size and shape that fits most common portable electronic devices. In alternative embodiment, the sanitization section is smaller than the one shown in FIG. 3 and is of a size and shape to fit smaller electronic devices such as cellular phones and portable audio players. In FIG. 2, the sanitization chamber 10 has a generally rectangular shape. In alternative embodiments, the sanitization chamber can be of any shape which is best fits the object to be sanitized, including a generally square shape, a generally circular shape, a generally oval shape and a generally triangular shape.

The sanitization section 18 is also sized so as to accommodate the amount of gaseous chlorine dioxide vapour that will be produced in the well 16. Specifically, the sanitization section 18 is small enough so that the amount of gaseous chlorine dioxide vapour produced in the well 16 will be sufficient to cover the entire volume of the sanitization section 18.

The base 12 also has a pathway 20 extending between the well 16 and the sanitization section 18. The gaseous chlorine dioxide vapour is formed in the well 16, travels through the pathway 20, and into the sanitization section 18 where the object or objects to be sanitized are being held. The pathway 20 is preferably enclosed when the cover 14 is placed over the base 12 so as to prevent gaseous chlorine dioxide vapour from escaping before it enters into the sanitization section 18.

FIG. 2 illustrates the cover 14 placed over the base 12 to substantially enclose the sanitization chamber 10. The closure does not form an airtight or hermetic seal. As such, gas can pass out of the closed sanitization chamber 10. For example, gas can pass out a space between the base 12 and cover 14. Alternatively, gas can pass out a vent in the wall of the sanitization chamber 10. This prevents pressure inside the sanitization chamber 10 from substantially increasing. Pressure inside the sanitization chamber 10 is maintained at substantially atmospheric pressure, preferably so as to not exceed 0.2 psig above atmospheric pressure, more preferably so as to not exceed 0.1 psig above atmospheric pressure.

The cover 14 preferably provides a plurality of storage areas 22 which can be used to hold vials containing the chlorite source and the acid releasing agent. The chlorite source and the acid releasing agent are preferably stored in separate vials to prevent them from reacting with each other prior to using the sanitization chamber 10.

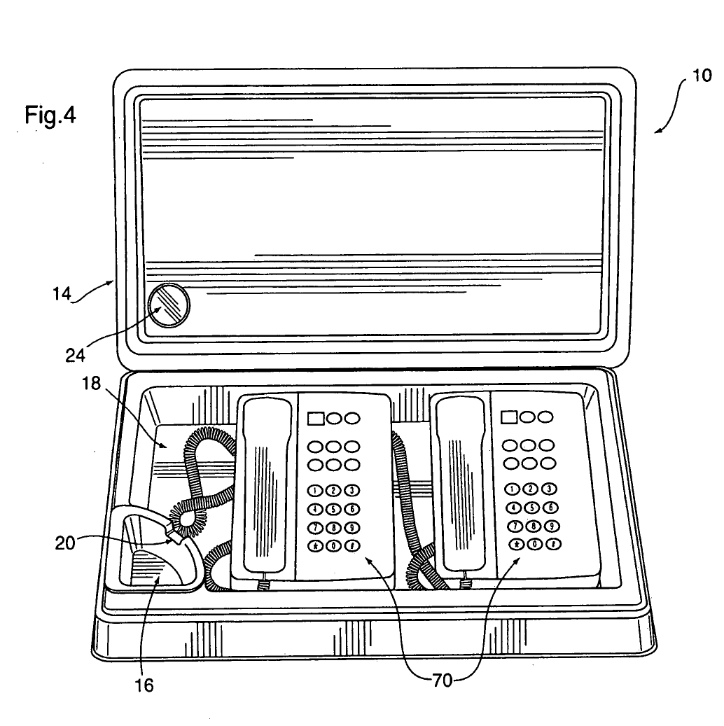

Furthermore, in a preferred embodiment, the cover 14 has a transparent viewing window 24, as shown in FIGS. 3 and 4. A user can see into the interior of the sanitization chamber 10 through the transparent viewing window 24 after the cover 14 has been placed over the base 12.

The chlorite source may be in the form of a gas, a liquid or a solid. Preferably, the chlorite source is in the form of a solid dry powder. The acid releasing agent may also be in the form of a gas, a liquid or a solid. Preferably, the acid releasing agent is in the form of a solid dry powder.

The chlorite source can be any compound which provides chlorite anions to form chlorine dioxide when acidified. Examples of suitable chlorite sources are alkali metal chlorites, alkaline-earth metal chlorites and chlorite salts of a transition metal ion or a protonated primary, secondary, tertiary or quaternary amine. Specific chlorite compounds which can act as the chlorite source in the present invention include, but are not limited to, sodium chlorite, potassium chlorite, calcium chlorite, ammonium chlorite, trialkylammonium chlorite and quarternary ammonium chlorite. Sodium chlorite is a preferred chlorite source. It is more preferred to use a dry powder of sodium chlorite as the chlorite source.

The acid releasing agent can be any acid or substance that can be hydrolized to an acid which is capable of reacting with the chlorite source to form chlorine dioxide. Examples of acid releasing agents which can be used in the present invention include, but are limited to, carboxylic acids, anhydrides, acyl halides, phosphoric acid, phosphate esters, trialkylsilyl phosphate esters, dialkyl phosphates, poly phosphates, condensed phosphates, sulfonic acid, sulfonic acid esters, sulfonic acid chlorides, phosphosilicates, phosphosilicic anhydrides, carboxylates of poly α-hydroxy alcohols, phosphosiloxanes, hydrochloric acid, boric acid, citric acid, malic acid, tartaric acid, and mineral acids. Sodium bisulphate is a preferred acid releasing agent for use in the present invention. It is more preferred to use a dry powder of sodium bisulphate in the present invention.

A chlorite source and an acid releasing agent which can be used in the sanitization process and with the sanitization chamber of the present invention can, for example, be a dry powder of sodium chlorite and a dry powder of sodium bisulphate manufactured by GO₂ International™ whose corporate headquarters are located in Westlake Village, Calif., United States. The dry powder of sodium chlorite is called GO₂ Component A™ and the dry powder of sodium bisulfate is called GO₂ Component B™. The dry powders manufactured by GO₂ International™ are generally used for treating water to provide safe drinking water, irrigation water and wash water, as well as to make a pressurized liquid spray for cleaning moulds in large buildings.

All of the reactants, namely the chlorite source, the acid releasing agent, and the water, are preferably provided in pre-measured amounts so that the reaction will efficiently produce gaseous chlorine dioxide vapour, and when the reaction is completed, there is only a minimal amount or no amount of chlorite source and acid releasing agent remaining. In a preferred embodiment, the dry powder of sodium chlorite and the dry powder of sodium bisulphate are provided in separate vials and are also provided in pre-measured amounts which will react with 100 mL of water in the well 16 to efficiently produce gaseous chlorine dioxide vapour, and when the reaction is completed, there is only a minimal amount or no amount of sodium chlorite powder and sodium bisulfate powder remaining.

FIGS. 3 and 4 illustrate examples of how the sanitization chamber 10 can be used. FIG. 3 illustrates the sanitization chamber 10 being used for sanitizing a computer keyboard 50 and a computer mouse 60. FIG. 4 illustrates the use of the sanitization chamber 10 for sanitizing two telephones 70.

In FIG. 3, the sanitization chamber 10 has been left in an open position and the cover 14 has not yet been placed onto the base 12. The computer keyboard 50 and the computer mouse 60 are placed into the sanitization section 18 of the base 12. The computer keyboard 50 and the computer mouse 60 do not need to be disconnected from the computer. As shown in FIG. 3, wires extend from the computer keyboard 50 and the computer mouse 60 to outside the sanitization chamber 10.

In a preferred embodiment, a dry powder of sodium chlorite, a dry powder of sodium bisulphate, and 100 mL of hot water are mixed in the well 16 to begin forming a gaseous chlorine dioxide vapour. The cover 14 is then placed over the base 12 to close the sanitization chamber 10, without sealing the sanitization chamber 10. Gases can pass outside the sanitization chamber 10, such as through a space between the base 12 and cover 14 or through a vent. The closed position of the sanitization chamber 10 can be seen in FIG. 2. The gaseous chlorine dioxide vapour formed in the well 16 travels through the pathway 20 and into the sanitization section 18. The gaseous chlorine dioxide vapour covers all surfaces of the computer keyboard 50 and the computer mouse 60, including hard to reach surfaces such as interior surfaces and surfaces between keyboard buttons. The gaseous chlorine dioxide vapour kills all pathogenic microorganisms including viruses, bacteria, fungi, protozoa, parasites and prions, on each surface of the computer keyboard 50 and the computer mouse 60.

A user should preferably allow at least one hour, and more preferably at least 6 hours, for the entire sanitization cycle to take place. After the sanitization cycle has been completed, the user can remove the cover 14, remove the sanitized computer keyboard 50 and the computer mouse 60, and dispose of any remaining solution in the well 16. The sanitization chamber 10 can then be stored away for future use.

In FIG. 4, the sanitization chamber 10 has also been left in an open position, and the cover 14 has not yet been placed onto the base 12. The two telephones 70 are placed into the sanitization section 18 of the base 12. The dry powder of sodium chlorite, the dry powder of sodium bisulphate, and the hot water are mixed in the well 16 to begin forming a gaseous chlorine dioxide vapour. The cover 14 is placed over the base 12 to substantially enclose the sanitization chamber 10. The closure is non-sealing, and gas can pass out of the sanitization chamber 10 through a space between the base 12 and the cover 14. The substantially enclosed position of the sanitization chamber 10 can be seen in FIG. 2. The gaseous chlorine dioxide vapour travels from the well 16, through the pathway 20, and into the sanitization section 18 where it covers all surfaces of the telephones 70, including interior surfaces, and kills microbes such as viruses, bacteria, fungi, protozoa, parasites and prions.

When placed into position over the base 12, the cover 14 does not form an airtight or hermetical sealing closure of the sanitization chamber 10. There is no substantial increase in pressure in the sanitization chamber 10 during use. The gaseous chlorine dioxide vapour is heavier than air and therefore, will tend to rest on the object to be sanitized. A small amount of gas may pass out of the sanitization chamber 10, such as through a space between the base 12 and the cover 14, thus preventing a substantial increase in pressure in the sanitization chamber 10. In another embodiment, gases can pass out a vent in the wall of the sanitization chamber 10. The closure being without a hermetical seal helps to avoid the substantial build-up of pressure in the sanitization chamber 10 and resulting explosions.

The sanitization process and the sanitization chamber 10 of the present invention are effective for killing pathogenic microorganisms, including viruses, bacteria, fungi, protozoa, parasites and prions which contaminate objects. For example, the sanitization process and the sanitization chamber 10 of the present invention are particularly effective for killing pathogenic microorganisms which contaminate electronic devices.

Examples of bacteria which have been shown to be killed by the chlorine dioxide vapour used in the present invention include, but are not limited to Pseudomonas Aeroginosa, Pseudomona Specie, Enterobarcer Cloaceae, Enerobarcter Hafina, Proteus Vulgaris, Klebsiella Pneumoniae, Salmonella Typhi, Salmonella Enteritidis, Salmonella Gallinarum, Salmonella Typhimorium, Salmonella Choleraesuis, Salmonella Typhosa, Corynebacterium Nucleatum, Sarcinae Lutae, Streptococus Pyrogenes, Strep 1,2,3, Mycobacterium Smegmatis, Campylobacter Jejuni, Flavobacterium Species, Yersinia Enterolitics, Clostridium Sporogenus, Clostridium Dificile, Clostridium Perfingens, Fusobacterium Nucleatum, Bacilus Subtilis, Bacilus Cerculans, Bacilus Meatarium, Bacilus Cereus, Bifedibacter Liberium, Staphylococcus Aureus, Staphylococcus epidermis, Streptococcus Faecalis, Mycobacterol Bovis, and Mycobacterium Kansaail.

Examples of viruses which have been shown to be killed by the chlorine dioxide vapour used in the present invention include, but are not limited to Herpes Virus I, Herpes Virus II, Adenovirus Echovirus, Coxakievirus, Influenza, Feline Parvovirus, Mouse Flu, Minute Virus of Mice (MVM), New Castle Disease Virus, Iridovirus, Poliovirus, Encephalomyocerditis (EMS), Vaccine Virus, Vasicular Stomatitis Virus (VSV), Para Influenza, Bluetongue Virus, Mouse Hepatitis Virus (MHV), Mouse Encephalomyelitis Virus, Mouse Polio Virus (MEV), and Pertiviries-Togaviridae.

Examples of fungi which have been shown to be killed by the chlorine dioxide vapour used in the present invention include, but are not limited to Candida Albicans, Scopulariosis Species, Trichophyton Mentagrophytes, Mucor Species, Saahromyces Cervisiae, Trichophyton Rubrum, Aspergillus Niger, Aspergillus Flavus, Fusarium Specie, and Fonsecaea Pedrosoi.

Other microorganisms which have been shown to be killed by the chlorine dioxide vapour used in the present invention include, but are not limited to Virio Cholerae, Culex Quinquifasiatus, and Mycoplasm.

The sanitization process and the sanitization chamber 10 of the present invention kill all pathogenic microorganisms which contaminate the objects being held in the sanitization section 18 of the sanitization chamber 10. As such, the microorganisms do not build up resistance to this sanitization process.

Advantageously, the sanitization process and the sanitization chamber 10 for carrying out this process of the present invention does not cause any damage to the object being treated. In particular, the sanitization process does not damage the electronic circuitry of electronic devices. Electronic devices can continue to run and operate while going through a sanitization cycle in the sanitization chamber 10.

The sanitization process and the sanitization chamber 10 are safe and easy to use. The chlorite source and the acid releasing agent are preferably provided in pre-measured amounts in separate vials. Therefore, a user simply needs to mix the pre-measured amounts of the chlorite source and the acid releasing agent with a set volume of hot water in the well 16 to produce the gaseous chlorine dioxide vapour. A visible line can be formed or drawn in the side of the well 16 to indicate to a user the appropriate amount of hot water to be added to the well 16. For example, the visible line can be used to mark a volume of 100 mL. The user simply needs to then place the cover 14 over the base 12 and allow the sanitization cycle to take place.

The sanitization chamber 10 is light in weight, and portable. The vials containing the chlorite source and the acid releasing agent are also safe and easy to transport. The sodium chlorite dry powder and the sodium bisulphate dry powder are classified as an irritant, but they are not dangerous goods. These powders are packaged in small quantities so as to pose little or no threat to human health or safety in case of a packaging failure. This is confirmed in Material Safety Data Sheets prepared by GO₂ International™ for: (i) a powder composition containing sodium chlorite (CAS 7758-19-2); and (ii) a powder composition containing sodium bisulfate anhydrous (CAS 7681-38-1).

The sodium chlorite dry powder and the sodium bisulphate dry powder have a long shelf life of approximately five years or more. Therefore, there is no requirement to open the vials and use the dry powders until the user desires to carry out a sanitization cycle.

The sanitization process of the present invention does not result in side chemical reactions to produce undesirable or harmful by-products. For example, there is no chlorination of any organic materials. Also, no trihalomethanes, haloacetic acids, or other organic, carsinogenic, chlorophenal or estrogenic compounds are formed. The sodium chlorite dry powder and the sodium bisulphate dry powder do not tend to react with ammonia, ammonium or other organic compounds, unlike chlorine. As such, undesirable by-products which are harmful to human health are not produced.

The gaseous chlorine dioxide vapour produced in the sanitization process of the present invention is better suited than liquid sanitizers for use in applications such as sanitizing electronic devices, including computer keyboards, cellular phones, laptop computers, handheld video game devices, portable audio players, portable video players, and global positioning system devices. Unlike liquid sanitizers, the gaseous chlorine dioxide vapour does not damage the electronic circuitry of the electronic devices.

Although this disclosure has described and illustrated preferred embodiments of the invention, it is to be understood that the invention is not restricted to these particular embodiments. Rather, the invention includes all embodiments that are functional or mechanical equivalents of the specific embodiments and features that have been described and illustrated herein. Many modifications will now occur to those skilled in the art. For a definition of the invention, reference is made to the following claims.

We claim:

1. A process for sanitizing an object, the process comprising:
(i) placing the object to be sanitized in a substantially enclosed environment, the environment being at atmospheric pressure when the object is placed therein,
(ii) providing a gaseous chlorine dioxide vapour in the environment, and
(iii) exposing the gaseous chlorine dioxide vapour to the object to sanitize the object;
including maintaining a pressure in the environment at substantially atmospheric pressure.

2. The process of claim 1, wherein the environment is substantially enclosed such that gases in the environment can pass outside the environment to maintain the pressure in the environment at substantially atmospheric pressure.

3. The process of claim 2, wherein the pressure in the environment is maintained so as to not exceed 0.1 prig above atmospheric pressure.

4. The process of claim 3, wherein the object is an electronic device comprising an electronic circuit.

5. The process of claim 4, including operating the electronic circuit during the process.

6. The process of claim 4, wherein the electronic device is selected from the group consisting of a computer keyboard, a laptop computer, a cellular phone, a handheld video game device, a portable audio player, a portable video player, and a global positioning system device.

7. The process of claim 4, wherein the step of providing the gaseous chlorine dioxide vapour comprises reacting a chlorite source with an acid releasing agent.

8. The process of claim 7, wherein the chlorite source is sodium chlorite and the acid releasing agent is sodium bisulfate.

9. The process of claim 8, wherein the sodium chlorite is in a form of a dry powder and the sodium bisulfate is in a form of a dry powder, and
the dry powder of sodium chlorite and the dry powder of sodium bisulfate are mixed with water to form the gaseous chlorine dioxide vapour.

10. The process of claim 4, wherein the environment comprises a reaction section, a sanitization section, and a pathway extending between the reaction section and the sanitization section,
the step of placing the object in the environment comprising placing the object in the sanitization section,
the step of providing the gaseous chlorine dioxide vapour comprising providing the gaseous chlorine dioxide vapour in the reaction section, and
the step of exposing the gaseous chlorine dioxide vapour to the object comprising passing the gaseous chlorine dioxide vapour from the reaction section through the pathway, and into the sanitization section.

11. A sanitization chamber for sanitizing an object, the sanitization chamber being substantially enclosed, the sanitization chamber comprising:
(i) a reaction section where a gaseous chlorine dioxide vapour is generated, and
(ii) a sanitization section where the object to be sanitized is placed, the object being exposed to the gaseous chlorine dioxide vapour in the sanitization section,
a pressure in the sanitization chamber being at atmospheric pressure when the object is placed therein, and the pressure in the sanitization chamber being maintained at substantially atmospheric pressure during the sanitizing of the object.

12. The sanitization chamber of claim 11, wherein the sanitization chamber is substantially enclosed such that gases in the sanitization chamber can pass outside the sanitization chamber to maintain the pressure in the sanitization chamber at substantially atmospheric pressure.

13. The sanitization chamber of claim 12, wherein the pressure in the sanitization chamber is maintained so as to not exceed 0.1 psig above atmospheric pressure

14. The sanitization chamber of claim 13, wherein the object is an electronic device comprising an electronic circuit.

15. The sanitization chamber of claim 14, wherein the electronic circuit operates during the sanitizing of the electronic device.

16. The sanitization chamber of claim 14, wherein the electronic device is selected from the group consisting of a computer keyboard, a laptop computer, a cellular phone, a handheld video game device, a portable audio player, a portable video player, and a global positioning system device.

17. The sanitization chamber of claim 14, wherein the gaseous chlorine dioxide vapour is generated by reacting sodium chlorite with sodium bisulfate in the reaction section.

18. The sanitization chamber of claim 17, wherein the sodium chlorite is in a form of a dry powder and the sodium bisulfate is in a form of a dry powder, and
the dry powder of sodium chlorite and the dry powder of sodium bisulfate are mixed with water in the reaction section to generate the gaseous chlorine dioxide vapour.

19. The sanitization chamber of claim 14, further comprising a pathway extending between the reaction section and the sanitization section, the gaseous chlorine dioxide vapour passing from the reaction section through the pathway and into the sanitization section where the object is held.

20. The sanitization chamber of claim 19, wherein the sanitization chamber comprises a base and a cover,
the reaction section, the sanitization section and the pathway being formed in the base, and
the cover being placed over the base when the generating of the gaseous chlorine dioxide vapour begins in the reaction section, whereby the cover substantially encloses the sanitization chamber.
